(19) United States
(12) Patent Application Publication
(10) Pub. No.: US 2026/0343171 A1
(43) Pub. Date: Oct. 29, 2026

(54) SOUND STIMULUS GENERATING APPARATUS, SOUND STIMULUS GENERATING METHOD, AND PROGRAM

(71) Applicant: NTT, Inc., Tokyo (JP)

(72) Inventors: Hiroaki ITO, Musashino-shi, Tokyo (JP); Kenichi NOGUCHI, Musashino-shi, Tokyo (JP); Hironobu CHIBA, Musashino-shi, Tokyo (JP); Tatsuya KAKO, Musashino-shi, Tokyo (JP)

(73) Assignee: NTT, Inc., Tokyo (JP)

(21) Appl. No.: 19/106,207

(22) PCT Filed: Aug. 30, 2022
(86) PCT No.: PCT/JP2022/032535

§ 371 Date: Feb. 25, 2025

(51) Int. Cl. H04S 7/00; G10K 15/04

(52) U.S. Cl. CPC H04S 7/307; G10K 15/04

(57) ABSTRACT

Provided is a sound stimulus generating apparatus or the like that generates a sound stimulus optimal for a brain wave state of a subject. A sound stimulus generating apparatus includes: a beat frequency determination unit that determines a candidate α of a beat frequency optimal for a subject by using a score obtained when a predetermined task is performed for the subject and a brain wave signal of the subject; and a binaural beat generation unit that generates a binaural beat signal by using the candidate α.

### TECHNICAL FIELD

The present invention relates to a technique of generating a binaural beat signal.

### BACKGROUND ART

There are conventional techniques that present an artificial auditory stimulus to encourage continued concentration. For example, Non Patent Literature 1 discloses that, when two pure sounds having slightly different frequencies called a binaural beat are presented to both ears and a beat sound (a groan sound) corresponding to the frequency difference is presented, brain waves are synchronized to activate concentration. When an angular frequency of a reference sound is represented by ω, a signal x_{L}(t)=sin(ω+α)t to which an angular frequency α is added is presented to the left ear, and a signal x_{R}(t)=sin(ω+α)t from which the angular frequency α is subtracted is presented to the right ear, listening is performed with the signals in a brain as an addition signal of y(t)=x_{L}(t)+x_{R}(t)=2 sin ωt cos αt.

This results in a waveform in which amplitude of the reference sound having the angular frequency ω is varied by a beat having the angular frequency α, and the beat presents a synchronization effect of brain waves. For example, in Non Patent Literature 2, a relationship between a beat frequency of a binaural beat to be presented and a frequency of a brain reaction is investigated, and it is confirmed that a brain wave band identical to a brain wave band of the beat frequency is emphasized, and it is further confirmed that attention during a task is improved on a next day subsequent to listening of the binaural beat.

### CITATION LIST

### Non Patent Literature

Non Patent Literature 1: Oster, “Auditory Beats in Brain,” Sci American, vol. 229, pp. 94-102, 1973.
Non Patent Literature 2: Ross et al., “40-Hz Binaural beats enhance training to mitigate the attentional blink.” Sci Report, vol. 10, pp. 7002, 2020.

### SUMMARY OF INVENTION

### Technical Problem

In the conventional techniques, it is assumed that identical brain waves to brain waves at a beat frequency are synchronized, and a fixed beat frequency is set for all subjects. That is, this is an argument that an equivalent effect is obtained by presenting a sound stimulus identical to all persons.

However, since there are individual differences among people, a desired effect (for example, an effect of improving concentration and attention) may not be obtained for a certain person.

An object of the present invention is to provide a sound stimulus generating apparatus, a sound stimulus generating method, and a program that generate a sound stimulus optimal for a brain wave state of a subject.

### Solution to Problem

To solve the problem described above, according to an aspect of the present invention, a sound stimulus generating apparatus includes: a beat frequency determination unit that determines a candidate α of a beat frequency optimal for a subject by using a score obtained when a predetermined task is performed for the subject and a brain wave signal of the subject; and a binaural beat generation unit that generates a binaural beat signal by using the candidate α.

### Advantageous Effects of Invention

According to the present invention, an effect is exerted that makes it possible to generate an optimal sound stimulus in consideration of an individual difference of a subject.

### BRIEF DESCRIPTION OF DRAWINGS

FIG. 1 is a functional block diagram of a sound stimulus generating apparatus according to first and second embodiments.

FIG. 2 is a diagram illustrating an example of a processing flow of the sound stimulus generating apparatus according to the first and second embodiments.

FIG. 3 is a diagram illustrating an image of processing of a beat frequency determination unit.

FIG. 4 is a functional block diagram of a binaural beat generation unit according to the second embodiment.

FIG. 5 is a diagram illustrating an example of a processing flow of the binaural beat generation unit according to the second embodiment.

FIG. 6 is a diagram illustrating an example of processing of the binaural beat generation unit according to the second embodiment.

FIG. 7 is a diagram illustrating a configuration example of a computer to which the present method is applied.

### DESCRIPTION OF EMBODIMENTS

Hereinafter, embodiments of the present invention will be described. Note that, in the drawings to be used in the description below, components having the same functions or steps for performing the same processing will be denoted by the same reference numerals, and redundant description will be omitted. In the following description, the symbol “-” or the like used in the text would normally be notated immediately above the immediately following character, but is notated immediately before the character due to limitations of text notation. In an expression, such a symbol is notated at the original position. In addition, processing performed in units of elements of a vector or a matrix is applied to all the elements of the vector or the matrix unless otherwise specified.

### Points of First Embodiment

In the present embodiment, from a brain wave signal of a subject and task performance data of the subject while a certain task is performed, a brain wave band in which power increases when the subject is exerting good performance is extracted and determined as a beat frequency candidate.

### First Embodiment

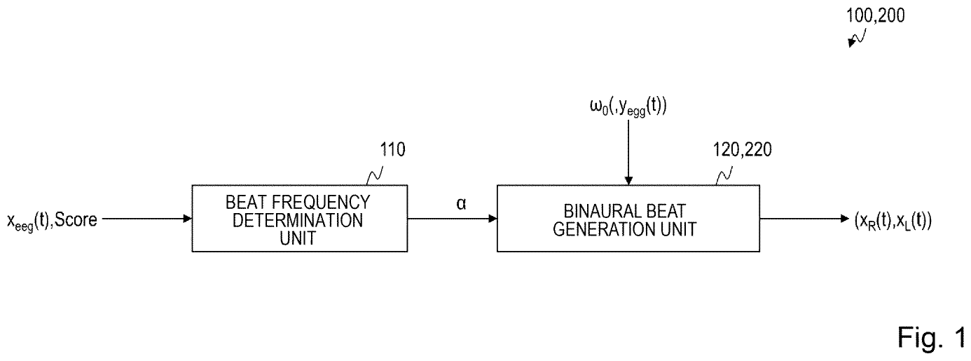

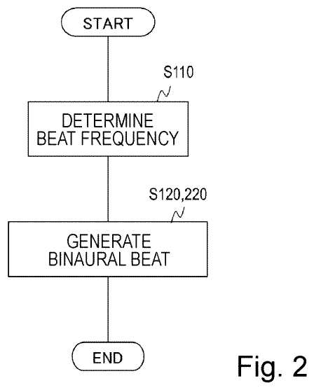

FIG. 1 illustrates a functional block diagram of a sound stimulus generating apparatus 100 according to the first embodiment, and FIG. 2 illustrates its processing flow.

The sound stimulus generating apparatus 100 includes a beat frequency determination unit 110 and a binaural beat generation unit 120.

The sound stimulus generating apparatus 100 receives, as input, a brain wave signal x_{eeg}(t) of the subject and a score Score (for example, time-series data of task performance) of the subject while a certain task is performed, determines an optimal beat frequency for obtaining a desired effect, generates a binaural beat signal (x_{L}(t), x_{R}(t)) corresponding to the optimal beat frequency, and outputs the generated binaural beat signal to a player including stereo speakers such as a pair of earphones or a headphone.

The sound stimulus generating apparatus 100 is a special device configured such that a special program is read by a known or dedicated computer having, for example, a central processing unit (CPU), a main storage device (random access memory (RAM)), and the like. The sound stimulus generating apparatus 100 executes each process under the control of the central processing unit, for example. Data input to the sound stimulus generating apparatus 100 or data obtained in each process is stored in, for example, a main storage device, and the data stored in the main storage device is read to the central processing unit as necessary and used for other processes. At least some of the processing units of the sound stimulus generating apparatus 100 may be configured by hardware such as an integrated circuit. Each of the storage units included in the sound stimulus generating apparatus 100 can be configured by, for example, a main storage device such as a random access memory (RAM) or middleware such as a relational database and a key value store. However, each storage unit is not necessarily provided inside the sound stimulus generating apparatus 100, and may be configured with an auxiliary storage device configured with a semiconductor memory element such as a hard disk, an optical disk, or a flash memory, and may be provided outside the sound stimulus generating apparatus 100.

Hereinafter, each of the units will be described.

### <Beat Frequency Determination Unit 110>

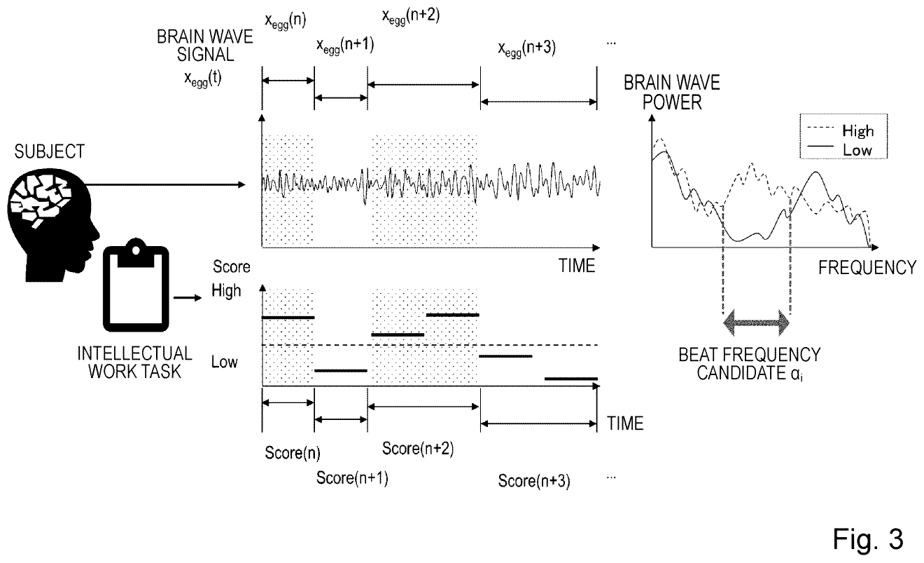

FIG. 3 is a diagram illustrating an image of processing of the beat frequency determination unit 110.

The beat frequency determination unit 110 receives, as input, the score Score obtained when a predetermined task is performed for the subject and the brain wave signal x_{eeg}(t) of the subject measured during the task, and using the input values, determines (S110) and outputs a candidate α of a beat frequency optimal for the subject. The t represents time.

The score Score represents task performance (time-series data) when a certain task is performed, and Score=(Score(1), . . . , Score(n), . . . , and Score(N)). For example, a task is a one-problem-to-one-answer task, and, task performance is indicated by using correct/incorrect determination Score_{Cor}(n)={True, False} of a problem, reaction time Score_{RT}(n) from when a problem is provided to when an answer is replied, and the like, where Score(n)=(Score_{Cor}(n), Score_{RT}(n)), Score_{RT}=(Score_{RT}(1), . . . , Score_{RT}(n), . . . , and Score_{RT}(N)), and Median (Score_{RT}) represents a median value of Score_{RT}. A larger value of Score_{RT}(n) indicates shorter reaction time. The n represents a problem number of a one-problem-to-one-answer task. Note that the number of problems of this task is N in total, and n=1, 2, . . . , and N. A task problem and a brain wave signal are associated with each other in advance, and a brain wave signal when the n-th task is performed is also referred to as x_{eeg}(n). Brain wave signals x_{eeg}(1), . . . , x_{eeg}(t), . . . , and x_{eeg}(T) include x_{eeg}(1), . . . , x_{eeg}(n), . . . , and x_{eeg}(N). T is time taken from when tasks are started to when all the tasks are ended.

For example, the beat frequency determination unit 110 extracts a brain wave signal x_{eeg}^{high}(n) when performance is high and a brain wave signal x_{eeg}^{low}(n) when performance is low from the brain wave signal x_{eeg}(t) using the score Score. For example, an equation described below is used to perform extraction.

$$\begin{matrix}[ Math . 1 ] & \\ \{ \begin{matrix}{x}_{eeg}^{high} ( n ) ← x ( n ) if {Score}_{RT} ( n ) \geq Median ( {Score}_{RT} ) and \\ {Score}_{Cor} ( n ) = True \\ {x}_{eeg}^{low} ( n ) ← x ( n ) if {Score}_{RT} ( n ) < Median ( {Score}_{RT} ) and \\ {Score}_{Cor} ( n ) = False\end{matrix} & ( 1 )\end{matrix}$$

In this example, the brain wave signal x_{eeg}^{high}(n) when performance is high is a brain wave signal when reaction time is equal to or less than a median value and a correct answer is replied, and the brain wave signal x_{eeg}^{low}(n) when performance is low is a brain wave signal when reaction time is longer than the median value and an incorrect answer is replied. Note that, as task performance, various indexes indicating a degree of performance can be used in addition to correct/incorrect determination of a problem and reaction time from when a problem is provided to when an answer is replied.

Next, the beat frequency determination unit 110 performs short-time Fourier analysis on the extracted brain wave signals x_{eeg}^{high}(n) and x_{eeg}^{low}(n), and calculates average values −x_{eeg}^{high}(f) and −x_{eeg}^{low}(f) of a power spectrum for each frequency f.

Finally, the beat frequency determination unit 110 compares the average values −x_{eeg}^{high}(f) and −x_{eeg}^{low}(f) for each frequency f, rearranges frequencies at which brain wave power (−x_{eeg}^{high}(f)) when performance is high exceeds the brain wave power (−x_{eeg}^{low}(f)) when performance is low in a descending order of the frequencies with larger power differences (−x_{eeg}^{high}(f)-−x_{eeg}^{low}(f)), and extracts and outputs a candidate α={α₁, α₂, . . . , and α_{L}} of a beat frequency.

$$\begin{matrix}[ Math . 2 ] & \\ {α}_{i} = \{ {f}_{i} \vert {\overline{X}}_{eeg}^{high} ( f ) > {\overline{X}}_{eeg}^{low} ( f ) \} & ( 2 )\end{matrix}$$

A candidate α is a vector in which L candidate values αᵢ of beat frequencies of binaural beats are stored. It is sufficient that L is any integer of 1 or more, and is set as appropriate. For example, L may be set so as to include all frequencies satisfying −x_{eeg}^{high}(f)-−x_{eeg}^{low}(f)>0, L may be set so as to include frequencies of top K% among frequencies satisfying −x_{eeg}^{high}(f)-−x_{eeg}^{low}(f)>0, L may be set, by providing a threshold Th, so as to include all frequencies satisfying −x_{eeg}^{high}(f)-−x_{eeg}^{low}(f)>Th, or L may be set to a predetermined integer and top L frequencies among frequencies satisfying −x_{eeg}^{high}(f)-−x_{eeg}^{low}(f)>0 may be extracted as a candidate α={α₁, α₂, . . . and α_{L}}. In the present embodiment, L=1.

### <Binaural Beat Generation Unit 120>

The binaural beat generation unit 120 receives, as input, a frequency ω₀ of the reference sound for a binaural beat and a beat frequency candidate α, and using the candidate α, generates (S120) and outputs a binaural beat signal (x_{L}(t), x_{R}(t)). The binaural beat signal (x_{L}(t), x_{R}(t)) is a signal corresponding to a brain wave state of the subject, is output to a player including stereo speakers such as a pair of earphones or a headphone, is played by the player, and is separately presented to ears of the subject. Note that the following expressions are satisfied.

x_{L}(t)=sin(ω₀+αₜ)(t)

x_{R}(t)=sin(ω₀−αₜ)(t)

Note that αₜ is an optimal beat frequency at time t, and, in the present embodiment, the beat frequency αₜ is a fixed value. For example, a frequency (in this example, α₁) presenting a maximum effect in a beat frequency candidate α is permanently set as a beat frequency.

### <Effects>

According to the present embodiment, a brain wave band identical to that of a candidate α is emphasized so that the subject is in a brain wave state of providing high performance (for example, a highly concentrated state). In addition, unlike the conventional techniques, it is possible to generate a sound stimulus optimized for an individual (a subject).

### Points of Second Embodiment

In the present embodiment, a binaural beat signal in which a beat frequency is optimized is generated on the basis of a beat frequency candidate and a brain wave state of the subject.

### Second Embodiment

Differences from the first embodiment will be mainly described.

FIG. 1 illustrates a functional block diagram of a sound stimulus generating apparatus 200 according to the second embodiment, and FIG. 2 illustrates its processing flow.

The sound stimulus generating apparatus 200 includes a beat frequency determination unit 110 and a binaural beat generation unit 220.

The sound stimulus generating apparatus 200 receives, as input, a brain wave signal x_{eeg}(t) of the subject and a score Score(for example, time-series data of task performance) of the subject while a certain task is performed, and determines a beat frequency candidate optimal for obtaining a desired effect. When a binaural beat is to be generated, the sound stimulus generating apparatus 200 receives, as an input value, a brain wave signal Y_{eeg}(t) of the subject when a binaural beat signal is presented, generates a binaural beat signal (x_{L}(t), x_{R}(t)) corresponding to an optimal beat frequency, and outputs the generated binaural beat signal to a player including stereo speakers such as a pair of earphones or a headphone.

Since processing of the beat frequency determination unit 110 is similar to that described in the first embodiment, description is omitted. However, in the present embodiment, L is any integer of 2 or more.

### <Binaural Beat Generation Unit 220>

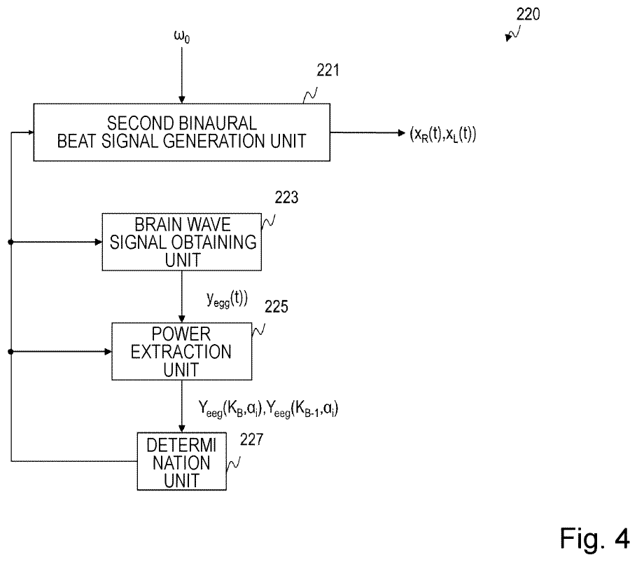

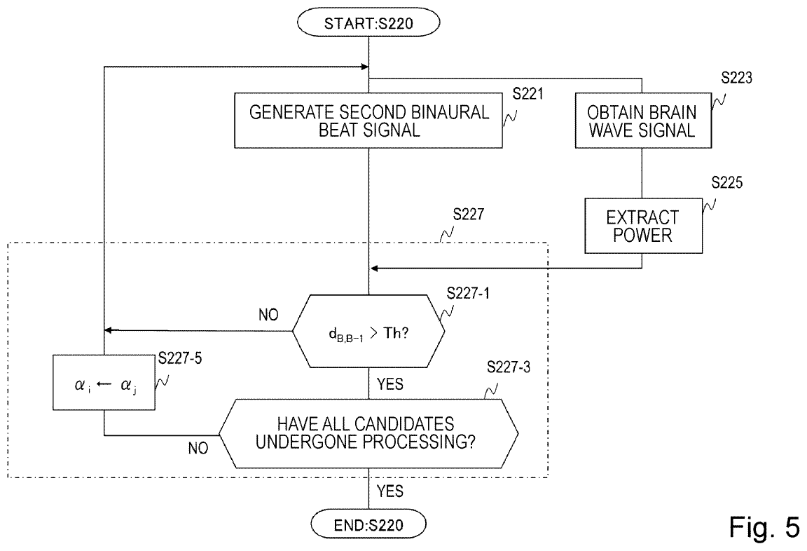

FIG. 4 illustrates a functional block diagram of the binaural beat generation unit 220 according to the second embodiment, and FIG. 5 illustrates its processing flow.

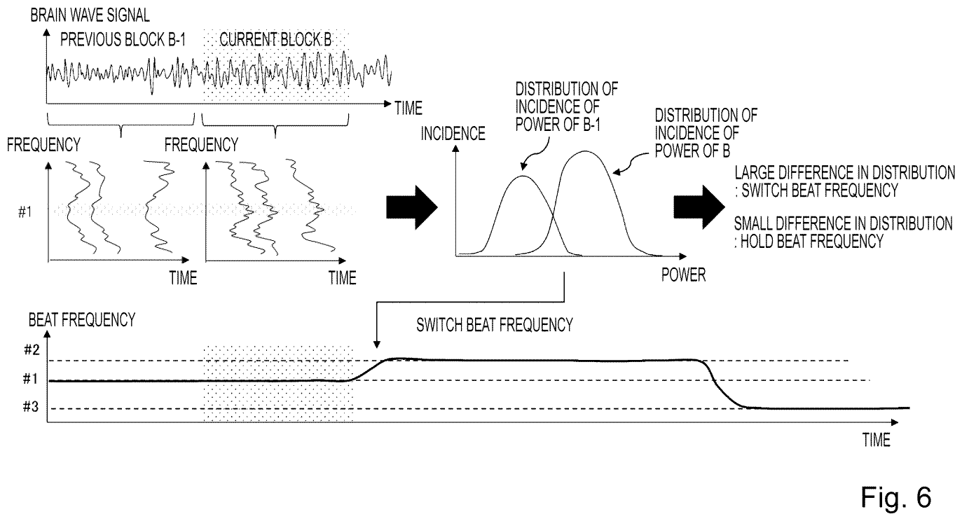

FIG. 6 is a diagram illustrating an image of processing of the binaural beat generation unit 220.

The binaural beat generation unit 220 includes a second binaural beat signal generation unit 221, a brain wave signal obtaining unit 223, a power extraction unit 225, and a determination unit 227.

Similar to the binaural beat generation unit 120, the binaural beat generation unit 220 also receives, as input, a frequency ω₀ of a reference sound for a binaural beat and a beat frequency candidate α, and using the candidate α, generates (S220) and outputs a binaural beat signal (x_{L}(t), x_{R}(t)). The binaural beat signal (x_{L}(t), x_{R}(t)) is a signal corresponding to a brain wave state of the subject, is output to a player including stereo speakers such as a pair of earphones or a headphone, is played by the player, and is separately presented to ears of the subject. While the beat frequency is a fixed value in the first embodiment, the beat frequency in the present embodiment is a variable. Hereinafter, processing of each unit will be described.

### (Second Binaural Beat Signal Generation Unit 221)

The binaural beat generation unit 221 receives, as input, a frequency ω₀ of the reference sound for a binaural beat and a beat frequency candidate α, and generates (S221) and outputs a binaural beat signal (x_{L}(t), x_{R}(t)) having a candidate αᵢ included in the candidate α as a beat frequency.

Note that the following expressions are satisfied.

x_{L}(t)=sin(ω₀+αᵢ)(t)

x_{R}(t)=sin(ω₀−αᵢ)(t)

In the present embodiment, the beat frequency αᵢ is a variable. For example, a frequency (in this example, αᵢ) presenting a maximum effect in the beat frequency candidate α is first used, and is changed in a descending order as α₂, . . . , and α_{L} from one presenting a largest effect to one presenting a smallest effect in accordance with control of the determination unit 227 described later.

### (Brain Wave Signal Obtaining Unit 223)

The brain wave signal obtaining unit 223 obtains and outputs a brain wave signal Y_{eeg}(t) of the subject to which the binaural beat signal (x_{L}(t), x_{R}(t)) is presented (S223).

For example, the brain wave signal obtaining unit 223 is an existing brain wave measurement device or an interface or the like that receives a brain wave signal measured by an ordinary brain wave measurement device.

### (Power Extraction Unit 225)

The power extraction unit 225 receives, as input, the brain wave signal y_{eeg}(t), converts the brain wave signal y_{eeg}(t) in a time domain into a signal in a frequency domain through short-time Fourier transform or the like, and extracts (S225) and outputs power Y_{eeg}(k_{B}, αᵢ) in a block section B and power Y_{eeg}(k_{B-1}, αᵢ) in a previous block section B-1. The block sections B and B-1 are time sections used when determination is performed by the determination unit 227 described later, and the block sections B and B-1 include N_{B} and N_{B-1} pieces of frames (frames when short-time Fourier transform or the like is to be performed), respectively, and the N_{B} and the N_{B-1} pieces of power at the frequency αᵢ are obtained for the block sections B and B-1, respectively. Indexes of frames included in the block sections B and B-1, respectively, are represented by k_{B} and k_{B-1}.

### (Determination Unit 227)

The determination unit 227 determines (S227-1) whether or not a brain wave band identical to a candidate di is emphasized from information based on a brain wave signal so that the subject is in a brain wave state of providing high performance, and, in a case where it is not emphasized (NO in S227-1), performs control to repeat processing S221, S223, and S225 using the candidate αᵢ, and, in a case where it is emphasized (YES in S227-1), selects (S227-5) a candidate αⱼ that has not been used for generation of a binaural beat signal from among L candidates αⱼ, and repeats the processing using the selected candidate αⱼ.

In the present embodiment, the determination unit 227 receives an Np pieces of power Y_{eeg}(k_{B}, αᵢ) and an N_{B-1} pieces of power Y_{eeg}(k_{B-1}, αᵢ), and calculates a difference d_{B,B-1} in distribution of incidence. The difference d_{B,B-1} in distribution of incidence corresponds to information based on a brain wave signal described above. Note that, as an amount indicating a difference in distribution of incidence, various values can be used, and for example, an effect amount such as Cohen's d (a value standardized by dividing a difference between average values by a sample standard deviation) expressed by an equation described below can be used.

$$\begin{matrix}[ Math . 3 ] & \\ {d}_{B , B - 1} = \frac{\vert {\overline{Y}}_{eeg} ( {k}_{B} , {α}_{i} ) - {\overline{Y}}_{eeg} ( {k}_{B - 1} , {α}_{i} ) \vert}{{s}_{c}} & ( 3 )\end{matrix}$$

$$\begin{matrix}[ Math . 4 ] & \\ {s}_{c} = \sqrt{\frac{{N}_{B} {S}_{B}^{2} + {N}_{B - 1} {S}_{B - 1}^{2}}{{N}_{B} + {N}_{B - 1}}} & ( 4 )\end{matrix}$$

Where −Y_{eeg}(k_{B}, αᵢ) and S_{B}² represent an average value and variance of the N_{B} pieces of power Y_{eeg}(k_{B}, αᵢ), respectively, and −Y_{eeg}(k_{B-1}, αᵢ) and S_{B-1}² represent an average value and variance of the N_{B-1} pieces of power Y_{eeg}(k_{B-1}, αᵢ), respectively.

The determination unit 227 determines whether or not an identical brain wave band to the candidate αᵢ is emphasized on the basis of a magnitude relationship between the difference d_{B,B-1} in distribution of incidence and the predetermined threshold Th (S227), and transmits a control signal to each unit in accordance with a result of the determination.

For example, in a case where a larger value d_{B,B-1} indicates a larger difference in distribution of incidence, the determination unit 227 performs processing as described below. When the difference d_{B,B-1} in distribution of incidence is equal to or less than the predetermined threshold Th (NO in S227-1), the determination unit 227 causes the second binaural beat signal generation unit 221 to perform processing using the candidate di used in the binaural beat generation unit 220, and outputs a control signal for performing control so as to repeat the processing in the brain wave signal obtaining unit 223 and the power extraction unit 225 to each unit.

When the difference d_{B,B-1} in distribution of incidence is larger than the predetermined threshold Th (YES in S227-1), the determination unit 227 determines whether or not all of the L candidates αᵢ have been used to generate a binaural beat signal, and, when all of the candidates αᵢ have been used (YES in S227-3), ends the processing. On the other hand, when there is a candidate αⱼ that has not been used to generate a binaural beat signal in the L candidates αᵢ (NO in S227-3), the determination unit 227 selects the candidate αⱼ that has not been used to generate a binaural beat signal from among the L candidates αᵢ (S227-5), performs processing in the second binaural beat signal generation unit 221 using the selected candidate αⱼ, and outputs a control signal for performing control so as to repeat the processing in the brain wave signal obtaining unit 223 and the power extraction unit 225 to each unit.

In this case, the fact that a difference in distribution of incidence is larger than the threshold means that a brain wave is emphasized in its frequency component. It is considered that, between a brain wave state of providing high performance and another brain wave state, a difference is not observed only in one frequency component, but differences are observed in a plurality of frequency components. In the first embodiment, a difference in one frequency component (for example, a frequency component that has presented a maximum effect) is focused on, and a brain wave is emphasized only for the frequency component. On the other hand, in the present embodiment, in order to bring a brain wave state closer to a brain wave state of providing high performance, a frequency component to be emphasized is changed each time an emphasis of a brain wave is confirmed for a plurality of frequency components presenting some effects, and brain waves are uniformly emphasized to aim for a higher effect. Note that a selection order of the candidates is not particularly limited. For example, the determination unit 227 may select the candidate in a descending order or an ascending order in accordance with a level of an effect, or may select the candidate randomly. However, it is considered that a brain wave state of a subject can be more promptly brought closer to a brain wave state of providing high performance by selecting the candidate in a descending order from one presenting a highest effect.

### <Effects>

With such a configuration, it is possible to obtain the same advantageous effects as those of the first embodiment. Furthermore, it is possible to bring a brain wave state closer to a brain wave state of providing high performance.

### Modification Example

Although, the present embodiment has a configuration in which the processing is ended in a case where all of the L candidates di are used for generating binaural beat signals (YES in S227-3), use states of the candidates may be reset, and processing of emphasizing a brain wave again may be repeated.

### Other Modification Examples

The present invention is not limited to the above-described embodiments and modification examples. For example, various kinds of processing described above may be executed not only in time series in accordance with the description but also in parallel or individually in accordance with processing abilities of the devices that execute the processing or as necessary. Modifications can be made as appropriate within the scope without departing from the concept of the present invention.

### <Program and Recording Medium>

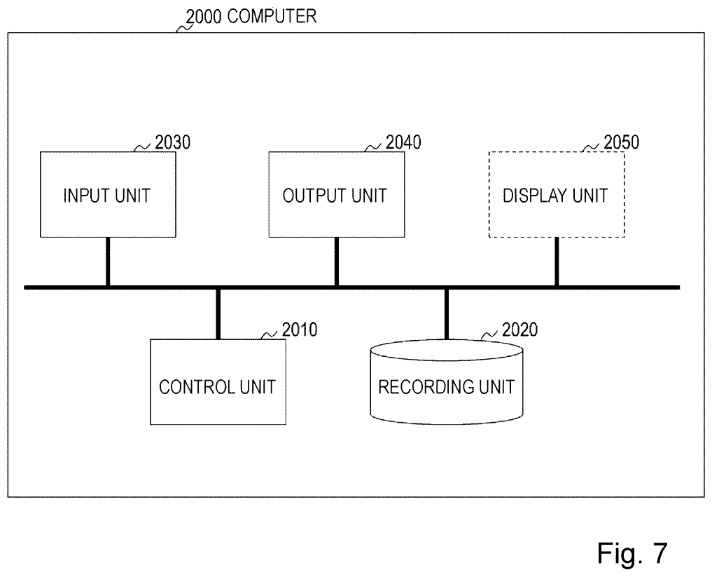

The various kinds of processing described above can be performed by causing a recording unit 2020 of a computer 2000 illustrated in FIG. 7 to read a program for executing each step of the method described above and causing a control unit 2010, an input unit 2030, an output unit 2040, a display unit 2050, and the like to operate the program.

The program in which the processing contents are described can be recorded on a computer-readable recording medium. The computer-readable recording medium may be, for example, any recording medium such as a magnetic recording device, an optical disc, a magneto-optical recording medium, or a semiconductor memory.

In addition, the program is distributed by, for example, selling, transferring, or renting a portable recording medium such as a DVD or CD-ROM in which the program is recorded. Furthermore, the program may be stored in a storage device of a server computer, and distributed by being transferred from the server computer to another computer via a network.

For example, a computer that executes such a program first temporarily stores a program recorded on a portable recording medium or a program transferred from a server computer in a storage device of its own. Then, when executing processing, the computer reads the program stored in the recording medium of its own and executes processing according to the read program. In addition, as another mode of executing the program, the computer may read the program directly from the portable recording medium and execute the processing according to the program, or may sequentially execute processing according to a received program every time the program is transferred from the server computer to the computer. In addition, the above-described processing may be executed by a so-called application service provider (ASP) type service that implements a processing function only by an execution instruction and result acquisition without transferring the program from the server computer to the computer. Note that the program in the present mode includes information that is used for processing by an electronic computing machine and is equivalent to the program (data or the like that is not a direct command to the computer but has a property that defines processing of the computer).

In addition, although the present device is configured by the predetermined program being executed on the computer in this mode, at least a part of the processing contents may be implemented by hardware.

1. A sound stimulus generating apparatus comprising:
processing circuitry configured to:
execute a beat frequency determination processing in which the processing circuitry determines a candidate α of a beat frequency optimal for a subject by using a score obtained when a predetermined task is performed for the subject and a brain wave signal of the subject; and
execute a binaural beat generation processing in which the processing circuitry generates a binaural beat signal by using the candidate α.

2. The sound stimulus generating apparatus according to claim 1, wherein
L is any integer of 1 or more, i is any one of 1, 2, . . . , and L, and the candidate α includes L candidates αᵢ, and
the binaural beat generation processing includes:
a second binaural beat signal generation processing in which the processing circuitry generates a binaural beat signal having one of the candidates αᵢ as a beat frequency;
a brain wave signal obtaining processing in which the processing circuitry obtains a brain wave signal of the subject to which the binaural beat signal that has been generated is presented; and
a determination processing in which the processing circuitry determines whether or not an identical brain wave band to a brain wave band of the one of the candidates αᵢ is emphasized from information based on the brain wave signal so that the subject is in a brain wave state of providing high performance, and in a case where the identical brain wave band is not emphasized, performs control to repeat processing using the one of the candidates αᵢ, and in a case where the identical brain wave band is emphasized, performs control to select a candidate αᵢ that has not been used for generation of a binaural beat signal from among the L candidates αᵢ and repeats the processing using the selected candidate αᵢ.

3. The sound stimulus generating apparatus according to claim 2, wherein
the binaural beat generation processing includes
a power extraction processing in which the processing circuitry extracts power Y_{eeg}(k_{B},αᵢ) in a block section B and power Y_{eeg}(k_{B-1},αᵢ) in a previous block section B-1 from the brain wave signal obtained by the brain wave signal obtaining processing,
in the determination processing the processing circuitry determines whether or not the identical brain wave band to the brain wave band of the one of the candidates αᵢ is emphasized on a basis of a magnitude relationship between a difference in distribution of incidence between the power Y_{eeg}(k_{B},αᵢ) and the power Y_{eeg}(k_{B-1},αᵢ) and a predetermined threshold, and
the information based on the brain wave signal is a difference in the distribution of incidence.

4. The sound stimulus generating apparatus according to claim 3, wherein
the difference in the distribution of incidence is a value standardized by dividing a difference between average values by a sample standard deviation.

5. The sound stimulus generating apparatus according to claim 1, wherein
the score includes correct/incorrect determination of a problem and reaction time from when the problem is provided to when an answer is replied.

6. A sound stimulus generating method, implemented by a sound stimulus generating apparatus that includes processing circuitry, comprising:
a beat frequency determination step in which the processing circuitry determines a candidate α of a beat frequency optimal for a subject by using a score obtained when a predetermined task is performed for the subject and a brain wave signal of the subject; and
a binaural beat generation step in which the processing circuitry of generating generates a binaural beat signal by using the candidate α.

7. A non-transitory computer-readable recording medium having recorded thereon a program for causing a computer to function as the sound stimulus generating apparatus according to claim 1.
